(19) United States
(12) Patent Application Publication
(10) Pub. No.: US 2011/0077153 A1
(43) Pub. Date: Mar. 31, 2011

(54) Substituted Heteroarylmethyl Sulfonamides

(75) Inventors: Jan Klaas Lohmann, Mannheim (DE); Alice Glaettli, Frankfurt (DE); Wassilios Grammenos, Ludwigshafen (DE); Bernd Mueller, Frankenthal (DE); Marianna Vrettou, Mannheim (DE); Jens Renner, Bad Duerkheim (DE); Sarah Ulmschneider, Bad Duerkheim (DE); Jochen Dietz, Karlsruhe (DE)

(73) Assignee: BASF SE, Ludwigshafen (DE)

(21) Appl. No.: 12/992,093

(22) PCT Filed: May 18, 2009
(86) PCT No.: PCT/EP09/55964

§ 371 Date: Nov. 11, 2010

(30) Foreign Application Priority Data
May 20, 2008 (EP) 08156504.6
Mar. 30, 2009 (EP) 09156739.6

(51) Int. Cl. A01C 1/06; C07D 239/34; A01N 43/54; C07D 237/16; A01N 43/58; A01P 3/00

(52) U.S. Cl. 504/100; 544/315; 514/274; 544/224; 514/247

(57) ABSTRACT

The present invention relates to heteroarylylmethyl sulfonamides of formula I

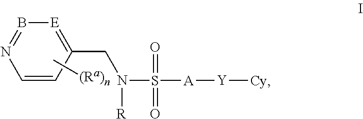

wherein B, E, R^{a}, n, R, A, Y and Cy are as defined in the claims, to the N-oxides, and salts thereof and their use for combating harmful fungi, and also to compositions and seed comprising at least one such compound. The invention also relates to a process and intermediates for preparing these compounds.

The present invention relates to compounds of formula I

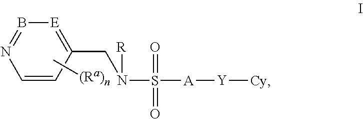

wherein:

B, E are N or CH or C—R^{a} with the provisos that one of B and E must be N and one of B and E must be CH or C—R^{a};

R^{a} is halogen, CN, NH₂, NO₂, OH, SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkyl-carbonyl, C₁-C₆-haloalkylcarbonyl, C₁-C₆-alkoxycarbonyl, C₁-C₆-haloalkoxy-carbonyl, C₁-C₆-alkylamino, C₁-C₆-haloalkylamino, di(C₁-C₆-alkyl)amino, di(C₁-C₆-haloalkyl)amino, C₁-C₆-alkylaminocarbonyl, di(C₁-C₆-alkyl)aminocarbonyl, C₁-C₆-alkoxy-C₁-C₆-alkyl, C₂-C₆-alkenyl, C₂-C₆-haloalkenyl, C₂-C₆-alkynyl, C₂-C₆-haloalkynyl, C₃-C₆-cycloalkyl or C₁-C₆-alkyl-C₃-C₈-cycloalkyl; and/or
two radicals R^{a} that are bound to adjacent ring member atoms of the pyridazine or pyrimidine ring may form together with said ring member atoms a fused 5-, 6- or 7-membered saturated, partially unsaturated or aromatic cycle, which may be a carbocycle or heterocycle, wherein the ring member atoms of the fused hetero-cycle include besides carbon atoms 1, 2, 3 or 4 heteroatoms selected from the group of N, O and S, and wherein the fused carbocycle or heterocycle is unsubstituted or carries 1, 2, 3 or 4 identical or different groups as defined for R^{a};

n indicates the number of the substituents R^{a} on the pyridazine or pyrimidine ring and n is 0, 1, 2 or 3, wherein R^{a} are identical or different if n is 2 or 3;

R is hydrogen, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylcarbonyl, C₁-C₆-haloalkyl-carbonyl, C₁-C₆-alkoxy-C₁-C₆-alkyl, C₁-C₆-haloalkoxy-C₁-C₆-alkyl, C₂-C₆-alkenyl, C₂-C₆-haloalkenyl, C₂-C₆-alkynyl, C₃-C₆-cycloalkyl, C₁-C₆-alkyl-C₃-C₆-cycloalkyl or benzyl, wherein the phenyl moiety of benzyl is unsubstituted or carries 1, 2, 3, 4, or 5 substituents selected from the group consisting of CN, halogen, C₁-C₆-alkyl, C₁-C₆-haloalkyl, C₁-C₆-alkoxy, C₁-C₆-haloalkoxy, C₁-C₆-alkylcarbonyl, C₁-C₆-halo-alkylcarbonyl, C₁-C₆-alkoxycarbonyl and di(C₁-C₆-alkyl)aminocarbonyl;

A is C₁-C₈-alkanediyl, C₁-C₆-haloalkanediyl, C₂-C₆-alkenediyl, C₂-C₆-haloalkenediyl, C₂-C₆-alkynediyl, C₂-C₆-haloalkynediyl, phenylene or a 5- or 6-membered heteroarenediyl, wherein the ring member atoms of the heteroarenediyl include besides carbon atoms 1, 2, 3 or 4 heteroatoms selected from the group of N, O and S, and wherein the aforementioned divalent radicals are unsubstituted or carry 1, 2, 3 or 4 identical or different groups R^{b}:
R^{b} is halogen, CN, NO₂, C₁-C₆-alkyl, C₁-C₆-haloalkyl, C₁-C₆-alkoxy, C₁-C₆-halo-alkoxy, C₂-C₆-alkenyl, C₂-C₆-haloalkenyl, C₂-C₆-alkynyl, C₂-C₆-haloalkynyl, (C₁-C₆-alkyl)carbonyl, (C₁-C₆-alkoxy)carbonyl, C₁-C₆-alkylamino, di(C₁-C₆-alkyl)amino, (C₁-C₆-alkyl)aminocarbonyl and di(C₁-C₆-alkyl)aminocarbonyl;
if A is a cyclic divalent radical, two radicals R^{b} that are bound to adjacent ring member atoms of the group A may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and wherein the fused carbocycle or heterocycle is unsubstituted or carries 1, 2, 3 or 4 identical or different groups as defined for R^{b};

Y is a divalent group selected from —O—, —OCH₂—, —CH₂O—, —S—, —S(═O)—, —S(═O)₂—, —N(R^{Π})— and —C(NOR^{Π})—;
R^{Π} is hydrogen or C₁-C₆-alkyl;

Cy is C₃-C₁₀-cycloalkyl or C₃-C₁₀-cycloalkenyl, which are unsubstituted or carry 1, 2, 3, 4 or 5 identical or different groups R^{c}:
R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkyl-sulfonyl, C₁-C₆-haloalkylsulfonyl, C₁-C₆-alkoxy-C₁-C₆-alkyl, C₁-C₆-halo-alkoxy-C₁-C₆-alkyl, C₂-C₆-alkenyl, C₂-C₆-alkynyl, C(═O)R′, C(═NOR″)R′″, C₃-C₈-cycloalkyl, C₁-C₆-alkyl-C₃-C₈-cycloalkyl, phenyl, phenoxy, phenoxy-C₁-C₆-alkyl or a 5- or 6-membered heteroaryl, wherein the ring member atoms of the heteroaryl include besides carbon atoms 1, 2, 3 or 4 heteroatoms selected from the group of N, O and S, and wherein the aforementioned cyclic radicals are unsubstituted or carry 1, 2, 3 or 4 identical or different substituents R^{d}:
   R′ is hydrogen, NH₂, C₁-C₆-alkyl, C₁-C₆-haloalkyl, C₂-C₆-alkenyl, C₂-C₆-alkynyl, C₁-C₆-alkoxy, C₁-C₆-alkoxy-C₁-C₆-alkoxy, C₁-C₆-halo-alkoxy, C₁-C₆-alkylamino or di(C₁-C₆-alkyl)amino;
   R″ is hydrogen, C₁-C₆-alkyl, C₁-C₆-haloalkyl, C₂-C₆-alkenyl, C₂-C₆-alkynyl or C₁-C₆-alkoxy-C₁-C₆-alkyl,
   R′″ is hydrogen or C₁-C₆-alkyl;
   R^{d} is halogen, CN, C₁-C₆-alkyl, C₁-C₆-haloalkyl, C₁-C₆-alkoxy or C₁-C₆-haloalkoxy;
   and/or two radicals R^{c} that are bound to adjacent ring member atoms of the group Cy may form together with said ring member atoms a fused 5-, 6- or 7-membered saturated, partially unsaturated or aromatic aromatic cycle, which may be a carbocycle or heterocycle, wherein the ring member atoms of the fused heterocycle include besides carbon atoms 1, 2, 3 or 4 heteroatoms selected from the group of N, O and S, and wherein the fused carbo-cycle or heterocycle is unsubstituted or carries 1, 2, 3 or 4 identical or different groups R^{e}:
   R^{e} is halogen, CN, C₁-C₆-alkyl, C₁-C₆-haloalkyl, C₁-C₆-alkoxy or C₁-C₆-haloalkoxy;
      and its N-oxides and agriculturally acceptable salts thereof.

The invention also relates to processes and intermediates for preparing such compounds, to agrochemical compositions comprising a solvent or solid carrier and at least a compound of formula I or an N-oxide or an agriculturally acceptable salt thereof and their use for combating phytopathogenic fungi, and seed comprising a compound of formula I, or an N-oxide or an agriculturally acceptable salt thereof.

WO 05/033081 describes pyridin-4-ylmethyl sulfonamides and their use for combating phytopathogenic fungi. The publication WO 06/097489 describes various pyridin-4-ylmethylamides of phenyl sulfonic acid and their use as fungicides.

The compounds according to the present invention differ from those described in WO 05/033081 and WO 06/097489 by having a pyridazinylmethyl or a pyrimidinylmathyl attached to the nitrogen of the sulfonamide group.

However, with respect to their fungicidal activity, the action of the known compounds is not always completely satisfactory. Based on this, it was an object of the present invention to provide compounds having improved action and/or a broadened activity spectrum against harmful fungi.

This object is achieved by substituted heteroarylmethyl sulfonamides of formula I and its N-oxides and their salts, in particular the agriculturally acceptable salts, as defined herein.

The compounds I can be prepared by various routes in analogy to prior art processes known per se for preparing sulfonamides and, advantageously, by the synthesis shown in the following schemes and in the experimental part of this application.

A further aspect of the present invention relates to a process for preparing compounds I as defined before, which comprises reacting compounds II, wherein B, E, R^{a}, n, and R are defined as above, under basic conditions with compounds III, wherein A, Y and Cy are defined as above and L is a nucleophilic leaving group such as halogen, substituted phenoxy, N₃, heterocyclyl or heterocyclyloxy, preferably pentafluorphenoxy, heterocyclyl such as imazolyl, pyrazolyl or triazolyl, or halogen such as chloro, fluoro or bromo, as shown below:

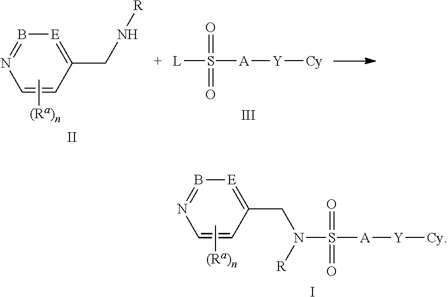

This reaction is usually carried out at temperatures of from −30 to 120° C., preferably from −10 to 100° C., in an inert organic solvent in the presence of a base.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as dichloromethane (DCM), chloroform and chlorobenzene, ethers, such as diethyl ether, diisopropyl ether, methyl tert.-butyl ether (MTBE), dioxane, anisole and tetrahydrofuran (THF), nitriles such as acetonitrile and propionitrile, ketones such as acetone, methyl ethyl ketone, diethyl ketone and tert.-butyl methyl ketone, and also dimethyl sulfoxide (DMSO), dimethyl formamide (DMF) and dimethyl acetamide, preferably THF, MTBE, dichloromethane, chloroform, acetonitrile, toluene or DMF, and also mixtures thereof.

Suitable bases are, in general, inorganic compounds such as alkali metal and alkaline earth metal hydroxides such as lithium hydroxide, sodium hydroxide, potassium hydroxide and calcium hydroxide, alkali metal and alkaline earth metal oxides such as lithium oxide, sodium oxide, calcium oxide and magnesium oxide, alkali metal and alkaline earth metal hydrides such as lithium hydride, sodium hydride, potassium hydride and calcium hydride, alkali metal and alkaline earth metal carbonates such as lithium carbonate, potassium carbonate and calcium carbonate, and also alkali metal bicarbonates such as sodium bicarbonate, moreover organic bases, e.g. tertiary amines such as trimethylamine, triethylamine, diisopropylethylamine and N-methylpiperidine (NMP), pyridine, substituted pyridines such as collidine, lutidine and 4-dimethylaminopyridine, and also bicyclic amines. Particular preference is given to triethylamine, pyridine, triethylamine and potassium carbonate. The bases are generally employed in catalytic amounts; however, they can also be used in equimolar amounts, in excess or, if appropriate, as solvent. The amount of base is typically 0.5 to 5 molar equivalents relative to 1 mole of compounds II.

The starting materials, i.e. compounds II and compounds III, are generally reacted with one another in equimolar amounts. In terms of yield it may be advantageous to employ an excess of compound II based on compound III.

Alternatively, compounds IV, wherein B, E, R^{a} and n are as defined above and L′ is a leaving group such as methylsulfonyl, toluenesulfonyl, hydroxyl or a group as defined for L in formula III, preferably, methylsulfonyl, toluenesulfonyl or halogen such as chloro, bromo and iodo, can be reacted with compounds III.a, wherein R, A, Y and Cy are as defined above, to obtain directly compounds I as shown below:

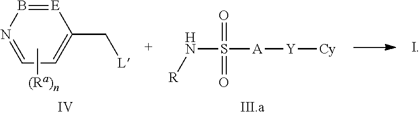

This reaction can be conducted under similar conditions as described for reacting compounds II with compounds III. Should other leaving groups L′ than hydroxy be desired, the hydroxy group can be effectively reacted to form the leaving group in question, e. g. in situ upon treatment with triphenylphosphine and diethylazodicarboxylate or diisopropylazodicarboxylate or a suitable substitute as described in Organ. Lett. 8, 5069-5072, 2006.

Alternatively, this reaction may also be carried out in two consecutive steps as shown below, wherein B, E, R^{a}, n, R, A, Y, Cy and L are defined as above:

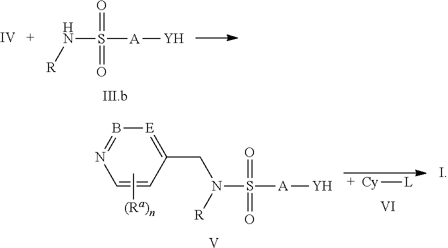

Both of the abovementioned reaction steps can be conducted under similar conditions as described for reacting compounds II with compounds III.

Alternatively, this reaction may also be carried as shown below, wherein B, E, R^{a}, n, R, A, Y, Cy and L are defined as above:

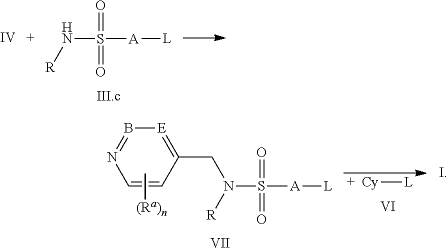

Both of the abovementioned reaction steps can be conducted under similar conditions as described for reacting compounds II with compounds III.

Alternatively, compounds I may also be obtained by first reacting compounds VIII, wherein A is as defined above and L¹ and L² are leaving goups and have one of the meanings mentioned for L in formula III, preferably being L¹ and L² different from each other, with compounds III to obtain compounds VII.a, which can be reacted with compounds VI.a to obtain compounds I as shown below:

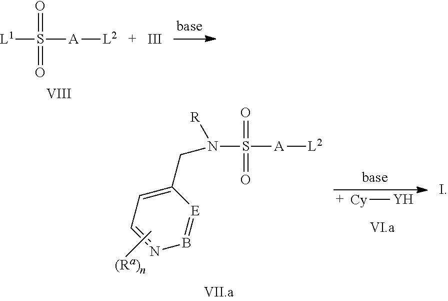

Both of the abovementioned reaction steps can be conducted under similar conditions as described for reacting compounds II with compounds III.

Some compounds II are known from the literature (cf. Bioorg. Med. Chem. 15(7), 2759-2767, 2007; U.S. 2007129547; WO 07/64993), are commercially available or they can be prepared by reactions known in the art e. g. by treatment with ammonia or ammonium acetate in the presence or absence of a suitable iodide salt, such as Nal, KI or tetrabutylammonium iodide, in an analogous fashion to the one described in WO 07/69685. Alternatively, compounds II may be prepared starting from derivatives IV by treatment with a suitable phthalimide salt, preferably K⁺ or Na⁺ salt, followed by hydrazine, as illustrated in U.S. 2007129547.

Alternatively, compounds II, wherein R is hydrogen, can be prepared by reduction of the corresponding oximes IX.a, nitriles IX.b, or amides IX.c or by reductive amination of the corresponding aldehydes IX.d or ketones IX.e as described below. Appropriate methods therefore are known to those skilled in the art:

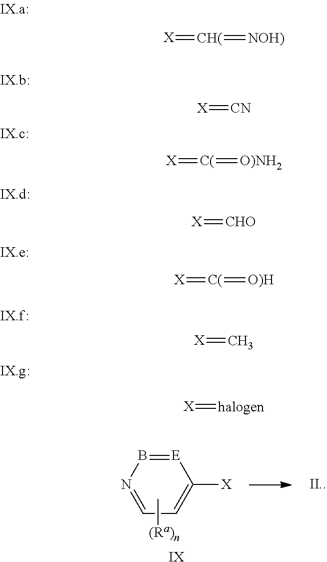

Methods suitable for the reduction of oximes IX.a, aldehydes IX.d or ketones IX.e to the corresponding compounds II have been described in the literature e.g. in March, J. “Advanced Organic Chemistry: Reactions, Mechanisms, and Structure” (Wiley & Sons, New York, 4th ed., 1992, pp. 1218-1219).

Methods suitable for the reduction of nitriles IX.b to the corresponding heteroaryl-methylamines II have been described in the literature, e.g. in March, J. “Advanced Organic Chemistry: Reactions, Mechanisms, and Structure” (Wiley & Sons, New York, 4th ed., 1992, 918-919).

Methods suitable for the reduction of amides IX.c to the corresponding compounds II have been described in the literature, e.g. in March, J. “Advanced Organic Chemistry: Reactions, Mechanisms, and Structure” (Wiley & Sons, New York, 4th ed., 1992, 1212-1213).

The oximes IX.a can be prepared prepared by reactions known in the art, e. g. from either the respective aldehydes IX.d, ketones IX.e, or the methyl derivatives IX.f in analogy to methods described by Houben-Weyl, vol. 10/4, Thieme, Stuttgart, 1968; vol. 11/2, 1957; vol E5, 1985; J. Prakt. Chem./Chem. Ztg. 336(8), 695-697, 1994; Tetrahedron Lett. 42(39), 6815-6818, 2001; Heterocycles 29(9), 1741-1760, 1989; or Liebigs Ann. Chem. 737, 39-45, 1970.

The aldehydes IX.d can be synthesized from the corresponding methyl derivatives IX.f in analogy to J. Org. Chem. 51(4), 536-537, 1986, or from halogenated derivatives IX.g as shown in Eur. J. Org. Chem. 2003(8), 1576-1588, 2003; Tetrahedron Lett. 40(19), 3719-3722 1999; or Tetrahedron 55(41), 12149-12156, 1999. The ketones IX.e may be prepared by oxidation of the corresponding alcohols using standard agents, e.g. in analogy to the methods described in Synthesis 11, 881-884; or Heterocycles 71(4), 911-918.

The nitriles IX.b can be prepared in analogy to methods described in Heterocycles, 41(4), 675 (1995); Chem. Pharm. Bull., 21, 1927 (1973); or J. Chem. Soc., 426 (1942); e.g. from the corresponding halogenated derivatives IX.g by reaction with cyanides such as CuCN, NaCN or KCN or in analogy to the route described in Monatsh. Chem. 87, 526-536, (1956), e.g. from the corresponding halogenated derivatives IX.g by reaction with a trialkylamine to afford the trialkylammonium substituted derivatives, followed by reaction with suitable cyanation reagents such as organic or inorganic cyanides, e.g. tetraalkylammonium cyanides, NaCN or KCN. The compounds IX.g are commercially available or can be synthesized according to standard methods.

The amides IX.c can be prepared, e.g. from the corresponding carboxylic acid chlorides or anhydrides by reaction with ammonia, e.g. as described in March, J. “Advanced Organic Chemistry: Reactions, Mechanisms, and Structure” (Wiley & Sons, New York, 3th edition, 1985, 370-371).

A further method to obtain compounds II is shown below, wherein PG is a suitable protection group that may be cleaved under acidic, basic or standard hydrogenation conditions such as defined below:

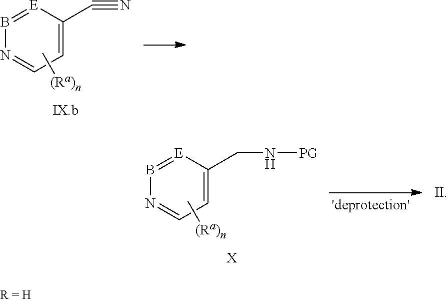

Protection of amino groups against reaction during one or more synthesis steps is a procedure well known and described in the art. Examples of suitable protection groups are those which are customarily used in organic synthesis, preferably t-butyloxy-carbonyl, benzyloxycarbonyl, allyloxy-carbonyl, diformyl or phthaloyl. Further details on suitable protection groups and their cleavage may be found in. Greene T. W., Wits P. G. “Protective groups in organic synthesis” (Wiley & Sons, New York, 1999, 494 et sqq.). The hydrogenation of the nitriles IX.b can be advantegously performed in the presence of suitable catalysts, preferably Raney nickel or palladium-on-carbon, and protection reagents such as di-tert.-butyl dicarbonate, dibenzyl dicarbonate, benzyl chloroformate, to yield the N-protected compounds X. On treating with hydrogen chloride or with hydrogen bromide/glacial acetic acid or with trifluoroacetic acid/water mixtures, the compounds X can be deprotected to yield compounds II, wherein R is hydrogen.

Compounds IV, wherein L′ is halogen, preferably Cl or Br, may be synthesized under standard halogenation conditions, e. g. by treatment of the corresponding methyl derivative IX.f with halogenation reagents such as Cl₂, Br₂, N-chlorosuccinimide, N-bromosuccinimide or isocyanuric chloride in analogy to methods described in Bioorg. Med. Chem. 15(10), 3315-3320; 2007, Eur. J. Org. Chem. 4, 947-957, 2006; J. Med. Chem. 48(5), 1367-1383, 2005; or J. Org. Chem. 68(11), 4179-4188, 2003.

Compounds IV, wherein L′ is methylsulfonyl or toluenesulfonyl, may be prepared under standard conditions by reacting the corresponding alcohol with methanesulfonic anhydride or trifluoromethanesulfonic anhydride, respectively, in analogy to methods described in J. Org. Chem. 50, 165-2170, 1985; or J. Chem. Soc. Perkin Trans. 1: Org. Bioorg. Chem. 12, 2887-2894, 1980.

The group R may be present in compounds II or may be introduced at a later stage as shown below by standard conditions in analogy to Coll. Czechoslovak. Chem. Comm. 40(4), 1193-1198, 1975 or J. Med. Chem. 19(12), 1409-1416, 1991, upon reaction of compounds I, wherein R is hydrogen, with suitable compounds XI, wherein the R and the leaving group L are as defined above and which compounds XI are known in the art:

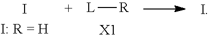

Compounds III and its derivatives III.a and III.b are known in the art and can be prepared in analogy to methods described in the European patent application 08101694.1.

If individual compounds I cannot be obtained by the routes described above, they can be prepared by derivatization of other compounds I.

The N-oxides may be prepared from the compounds I according to conventional oxidation methods, e. g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

If the synthesis yields mixtures of isomers, a separation is generally not necessarily required since in some cases the individual isomers can be interconverted during work-up for use or during application (e. g. under the action of light, acids or bases). Such conversions may also take place after use, e. g. in the treatment of plants in the treated plant, or in the harmful fungus to be controlled.

The term “compounds I” refers to compounds of formula I. Likewise, this terminology applies to all sub-formulae, e. g. “compounds I.A” refers to compounds of formula I.A or “compounds II” refers to compounds of formula II.

In the definitions of the variables given above, collective terms are used which are generally representative for the substituents in question. The term “Cₙ-Cₘ” indicates the number of carbon atoms possible in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-di-methylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Likewise, the term “C₁-C₄-alkyl” refers to a straight-chained or branched alkyl group having 1 to 4 carbon atoms.

The term “C₁-C₄-haloalkyl” refers to a straight-chained or branched alkyl group having 1 to 4 carbon atoms, wherein some or all of the hydrogen atoms in these groups may be replaced by halogen atoms, e. g. chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichioroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chloro-butyl, 4-bromobutyl or nonafluorobutyl. Likewise, the term “C₁-C₆-haloalkyl” refers to a straight-chained or branched alkyl group having 1 to 6 carbon atoms, wherein some or all of the hydrogen atoms in these groups may be replaced by halogen atoms.

The term “C₁-C₆-alkoxy” refers to a straight-chain or branched alkyl group having 1 to 4 carbon atoms which is bonded via an oxygen, at any position in the alkyl group, e.g. OCH₃, OCH₂CH₃, O(CH₂)₂CH₃, 1-methylethoxy, O(CH₂)₃CH₃, 1-methyl-propoxy, 2-methylpropoxy or 1,1-dimethylethoxy, O(CH₂)₄CH₃ or O(CH₂)5CH₃. Likewise, the term “C₁-C₄-alkoxy” refers to a straight-chain or branched alkyl group having 1 to 4 carbon atoms which is bonded via an oxygen, at any position in the alkyl group.

The term “C₁-C₄-haloalkoxy” refers to a C₁-C₄-alkoxy group, wherein some or all of the hydrogen atoms may be replaced by halogen atoms as mentioned above, e.g. OCH₂F, OCHF₂, OCF₃, OCH₂Cl, OCHCl₂, OCCl₃, chlorofluoromethoxy, dichlorofluoro-methoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloro⁻'ethoxy, OC₂F₅, 2-fluoropropoxy, 3-fluoropropoxy, 2,2-difluoropropoxy, 2,3-difluoro-propoxy, 2-chloro-propoxy, 3-chloropropoxy, 2,3-dichloropropoxy, 2-bromo-propoxy, 3-bromopropoxy, 3,3,3-trifluoropropoxy, 3,3,3-trichloropropoxy, OCH₂—C₂F₅, OCF₂—C₂F₅, 1-difluoromethyl-2-fluoroethoxy, 1-dichloromethyl -2-chloroethoxy, 1-dibromomethyl-2-bromo-ethoxy, 4-fluorobutoxy, 4-chlorobutoxy, 4-bromobutoxy or nonafluorobutoxy. Likewise, the term “C₁-C₆-haloalkoxy” refers to a C₁-C₆-alkoxy group, wherein some or all of the hydrogen atoms may be replaced by halogen atoms.

The term “C₁-C₄-alkoxy-C₁-C₄-alkyl” refers to alkyl having 1 to 4 carbon atoms, wherein one hydrogen atom of the alkyl radical is replaced by a C₁-C₄-alkoxy group. Likewise, the term “C₁-C₆-alkoxy-C₁-C₆-alkyl” refers to alkyl having 1 to 6 carbon atoms, wherein one hydrogen atom of the alkyl radical is replaced by a C₁-C₆-alkoxy group.

The term “C₁-C₄-haloalkoxy-C₁-C₄-alkyl” refers to alkyl having 1 to 4 carbon atoms, wherein one hydrogen atom of the alkyl radical is replaced by a C₁-C₄-haloalkoxy group. Likewise, the term “C₁-C₆-haloalkoxy-C₁-C₆-alkyl” refers to alkyl having 1 to 6 carbon atoms, wherein one hydrogen atom of the alkyl radical is replaced by a C₁-C₆-alkoxy group.

The term “C₁-C₆-alkoxy-C₁-C₆-alkoxy” refers to an C₁-C₆-alkoxy-C₁-C₆-alkyl group, which is bonded via an oxygen atom to the remainder of the molecule.

The term “C₁-C₄-alkylthio” as used herein refers to straight-chain or branched alkyl groups having 1 to 4 carbon atoms bonded via a sulfur atom, at any position in the alkyl group, e. g. methylthio, ethylthio, propylthio, isopropylthio, and n-butylthio. Likewise, the term “C₁-C₆-alkylthio” as used herein refers to straight-chain or branched alkyl groups having 1 to 6 carbon atoms bonded via a sulfur atom. Accordingly, the terms “C₁-C₄-haloalkylthio” and “C₁-C₆-haloalkylthio” refer to straight-chain or branched haloalkyl groups having 1 to 4 or 1 to 6 carbon atoms bonded through a sulfur atom, at any position in the haloalkyl group.

The terms “C₁-C₄-alkylsulfinyl” and “C₁-C₆-alkylsulfinyl”, respectively refer to straight-chain or branched alkyl groups having 1 to 4 or 1 to 6 carbon atoms, respectively, bonded through a —S(═O)- moiety, at any position in the alkyl group, e.g. methyl-sulfinyl and ethylsulfinyl, and the like. Accordingly, the terms “C₁-C₄-haloalkylsulfinyl” and “C₁-C₆-haloalkylsulfinyl”, respectively, refer to straight-chain or branched haloalkyl groups having 1 to 4 and 1 to 6 carbon atoms, respectively, bonded through a —S(═O)- moiety, at any position in the haloalkyl group.

The terms “C₁-C₄-alkylsulfonyl” and “C₁-C₆-alkylsulfonyl”, respectively, refer to straight-chain or branched alkyl groups having 1 to 4 and 1 to 6 carbon atoms, respectively, bonded through a —S(═O)₂- moiety, at any position in the alkyl group, e.g. methyl-sulfonyl. Accordingly, the terms “C₁-C₄-haloalkylsulfonyl” and “C₁-C₆-haloalkylsulfonyl”, respectively, refer to straight-chain or branched haloalkyl groups having 1 to 4 and 1 to 6 carbon atoms, respectively, bonded through a —S(═O)₂- moiety, at any position in the haloalkyl group.

The term “C₁-C₄-alkylamino” refers to an amino radical carrying one C₁-C₄-alkyl group as substituent, e.g. methylamino, ethylamino, propylamino, 1-methylethylamino, butylamino, 1-methylpropylamino, 2-methylpropylamino, 1,1-dimethylethylamino and the like. Likewise, the term “C₁-C₆-alkylamino” refers to an amino radical carrying one C₁-C₆-alkyl group as substituent.

The term “di(C₁-C₄-alkyl)amino” refers to an amino radical carrying two identical or different C₁-C₄-alkyl groups as substituents, e. g. dimethylamino, diethylamino, di-n-propylamino, diisopropylamino, N-ethyl-N-methylamino, N-(n-propyl)-N-methylamino, N-(isopropyl)-N methylamino, N-(n-butyl)-N-methylamino, N-(n-pentyl)-N-methylamino, N-(2-butyl)-N methylamino, N-(isobutyl)-N-methylamino, and the like. Likewise, the term “di(C₁-C₆-alkylamino” refers to an amino radical carrying two identical or different C₁-C₆-alkyl groups as substituents.

Accordingly, the terms “C₁-C₆-haloalkylamino” and “di(C₁-C₄-haloalkyl)amino”, respectively, refer to amino radicals carrying one and two identical or different C₁-C₆-alkyl groups as substituents, respectively.

The term “C₁-C₄-alkylcarbonyl” refers to a C₁-C₆-alkyl radical which is attached via a carbonyl group. The term “(C₁-Cₛ-alkoxy)carbonyl” refers to a C₁-C₆-alkoxy radical which is attached via a carbonyl group. Accordingly, the terms “C₁-C₆-haloalkylcarbonyl” and “C₁-C₆-haloalkoxycarbonyl”, respectively, refer to a C₁-C₆-alkyl radical and a C₁-C₆-alkoxy radical, respectively, which are attached via a carbonyl group.

The term “C₁-C₆-alkylaminocarbonyl” refers to a C₁-C₆-alkylamino radical which is attached via a carbonyl group. Likewise, the term “di(C₁-C₆-alkyl)aminocarbonyl” refers to a di(C₁-C₆)alkylamino radical which is attached via a carbonyl group.

The term “phenoxy” and refers to a phenyl radical which is attached via an oxygen atom. Likewise, the term “phenoxy-C₁-C₆-alkyl” and refers to a phenoxy radical which is attached via a C₁-C₆-alkyl group.

The term “C₂-C₄-alkenyl” refers to a straight-chain or branched unsaturated hydrocarbon radical having 2 to 4 carbon atoms and a double bond in any position, e.g. ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl. Likewise, the term “C₂-C₆-alkenyl” refers to a straight-chain or branched unsaturated hydrocarbon radical having 2 to 6 carbon atoms and a double bond in any position.

The term “C₂-C₄-alkynyl” refers to a straight-chain or branched unsaturated hydrocarbon radical having 2 to 4 carbon atoms and containing at least one triple bond, such as ethynyl, 1-propynyl, 2-propynyl, 1-butynyl, 2-butynyl, 3-butynyl, 1-methyl-2-propynyl.

Likewise, the term “C₂-C₆-alkynyl” refers to a straight-chain or branched unsaturated hydrocarbon radical having 2 to 6 carbon atoms and at least one triple bond.

The term “C₃-C₁₀-cycloalkyl” refers to monocyclic, bicyclic, bridged and diamandoid saturated hydrocarbon radicals having 3 to 10 carbon ring members, such as cyclopropyl, cyclobutyl, cyclopentyl, cyclohexyl, cycloheptyl, cyclooctyl, cyclononyl, cyclodecyl, norbornyl or adamantyl.

Likewise, the term “C₃-C₁₀-cycloalkenyl” refers to monocyclic, bicyclic and bridged unsaturated hydrocarbon radicals having 3 to 10 carbon ring members and a double bond in any position, such as cyclopropenyl, cyclobutenyl, cyclopentenyl, cyclohexenyl, cycloheptenyl, cyclooctenyl, cyclononenyl, cyclodecenyl or norbornenyl.

The term “C₁-C₆-alkyl-C₃-C₈-cycloalkyl” refers to a cycloalkyl radical having 3 to 8 carbon atoms (as defined above), wherein one hydrogen atom of the cycloalkyl radical is replaced by a C₁-C₆-alkyl group.

The term “5-, 6- or 7-membered carbocycle” is to be understood as meaning both saturated or partially unsaturated carbocycles having 5, 6 or 7 ring members as well as phenyl. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl, and the like.

The term “5-, 6-, or 7-membered heterocycle” wherein the ring member atoms of the heterocycle include besides carbon atoms one, two, three or four heteroatoms selected from the group of N, O and S, is to be understood as meaning both saturated and partially unsaturated as well as aromatic heterocycles having 5, 6 or 7 ring atoms.

### EXAMPLES INCLUDE

saturated and partially unsaturated 5-, 6-, or 7-membered heterocycle wherein the ring member atoms of the heterocycle include besides carbon atoms 1, 2 or 3 heteroatoms selected from the group of N, O and S, and which is saturated or partially unsaturated, e. g. pyrrolidin-2-yl, pyrrolidin-3-yl, tetrahydrofuran-2-yl, tetrahydrofuran-3-yl, tetrahydrothien-2-yl, tetrahydrothien-3-yl, 1,3-dioxolan-4-yl, isoxazolidin-3-yl, isoxazolidin-4-yl, isoxazolidin-5-yl, isothiazolidin-3-yl, isothiazolidin-4-yl, isothiazolidin-5-yl, pyrazolidin-3-yl, pyrazolidin-4-yl, pyrazolidin-5-yl, oxazolidin-2-yl, oxazolidin-4-yl, oxazolidin-5-yl, thiazolidin-2-yl, thiazolidin-4-yl, thiazolidin-5-yl, imidazolidin-2-yl, imidazolidin-4-yl, 2-pyrrolin-2-yl, 2-pyrrolin-3-yl, 3-pyrrolin-2-yl, 3-pyrrolin-3-yl, piperidin-2-yl, piperidin-3-yl, piperidin-4-yl, 1,3-dioxan-5-yl, tetrahydropyran-2-yl, tetrahydropyran-4-yl, tetrahydrothien-2-yl, hexahydropyridazin-3-yl, hexahydropyridazin-4-yl, hexahydropyrimidin-2-yl, hexahydropyrimidin-4-yl, 5-hexahydropyrimidinyl and piperazin-2-yl;
5-membered heteroaryl (heteroaromatic radical), wherein the ring member atoms of the heteroaryl include besides carbon atoms 1, 2 or 3 heteroatoms selected from the group of N, O and S, e. g. pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl, 1,2,4-thiadiazol-3-yl and 1,2,4-thiadiazol-5-yl;
6-membered heteroaryl (heteroaromatic radical), wherein the ring member atoms of the heteroaryl include besides carbon atoms 1, 2 or 3 heteroatoms selected from the group of N, O and S, e. g. pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl.

The term “C₁-C₆-alkanediyl” refers to a divalent, branched, or straight-chain saturated hydrocarbon radical having 1 to 4 carbon atoms, derived from a C₁-C₆-alkyl group that has two points of attachment.

Furthermore, the term “5- or 6-membered heteroarenediyl” refers to a divalent radical derived from a 5- or 6-membered heteroaryl having two points of attachment. Examples of heteroarenediyl radicals are, e.g. divalent radicals derived from pyridine, pyrimidine, pyridazine, 1,2,3-triazine, 1,2,4-triazine, 1,2,3,4-tetrazine, furan, thiophene, pyrrole, thiazole, thiadiazole, pyrazole, imidazole, triazole, tetrazole, oxazole, isoxazole, isothiazole, oxadiazole and the like. The aforementioned groups can be C-attached or N-attached where such is possible; e. g. a group derived from pyrrole, imidiazole or pyrazole can be N-attached or C-attached.

The term “phenylene” refers to 1,2-phenylene (o-phenylene), 1,3-phenylene (m-phenylene) and 1,4-phenylene (p-phenylene).

The term “two radicals R^{a} that are bound to adjacent ring member atoms of the pyridazine or pyrimidine ring may form together with said ring member atoms a fused 5-, 6- or 7-membered saturated, partially unsaturated or aromatic cycle ” refers to a condensed bicyclic ring system, wherein the pyridazine or pyrimidine ring carries a fused-on 5-, 6- or 7-membered carbocyclic or heterocyclic ring.

The term “two radicals R^{b} that are bound to adjacent ring member atoms of the group A may form together with said ring member atoms a fused 5-, 6- or 7-membered saturated, partially unsaturated or aromatic cycle” refers to a condensed bicyclic ring system, wherein the C₃-C₈-cycloalkylene, C₃-C₈-cycloalkenylene, phenylene or a 5- or 6-membered heteroarenediyl, respectively carry a fused-on 5-, 6- or 7-membered carbocyclic or heterocyclic ring.

The term “two radicals R^{c} that are bound to adjacent ring member atoms of the group Cy may form together with said ring member atoms a fused 5-, 6- or 7-membered saturated, partially unsaturated or aromatic aromatic cycle, which may be a carbocycle or heterocycle” refers to a condensed bicyclic ring system, wherein the C₃-C₈-cycloalkyl, phenyl and 5- or 6-membered heteroaryl, respectively carry a fused-on 5-, 6- or 7-membered carbocyclic or heterocyclic ring.

Agriculturally acceptable salts of compounds I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potasslum, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound of formula I with an acid of the corresponding anion, preferably of hydrochloric acid, hydrobromic acid, sulfuric acid, phosphoric acid or nitric acid.

The compounds of formula I can be present in atropisomers arising from restricted rotation about a single bond of asymmetric groups. They also form part of the subject matter of the present invention.

Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

In respect of the variables, the embodiments of the intermediates correspond to the embodiments of the compounds I.

Preference is given to those compounds I and where applicable also to compounds of all sub-formulae provided herein, e. g. formulae 1.1, 1.2, 1.3 and 1.4 or formulae I.A1 to I.F2 and to the intermediates such as compounds II, III, IV and IX.a to IX.h, wherein the substituents and variables (B, E, n, R, A, Y, Cy, R^{a}, R^{b}, R^{c}, R^{d}, R^{e}, Rⁿ, R′, R″ and R′″) have independently of each other or more preferably in combination the following meanings:

One embodiment of the invention relates to compounds I, wherein B is N and E is CH or C—R^{a}.

Another embodiment relates to compounds I, wherein B is CH or C—R^{a} and E is N.

One embodiment relates to compounds I, wherein n is 1 or 2, preferably n is 1. A further embodiment relates to compounds I, wherein n is 2 and R^{a} is in position 2 and 6 of the pyrimidine ring. A further embodiment relates to compounds I, wherein n is 2 and R^{a} is in position 3 and 6 of the pyridazine ring. A further embodiment relates to compounds I, wherein n is 0.

A further embodiment relates to compounds I, wherein two radicals R^{a} that are bound to adjacent ring member atoms of the pyridazine or pyrimidine ring do not form together with said ring member atoms any fused cycle.

In another embodiment, R^{a} is halogen, CN, C₁-C₄-alkyl, C₁-C₄-alkoxy, C₁-C₄-haloalkoxy, C₁-C₄-alkoxy-C₁-C₄-alkyl, C₃-C₈-cycloalkyl or C₁-C₄-alkyl-C₃-C₈-cycloalkyl.

In a further embodiment, R^{a} is CH₂CH₃, CH₂(CH₃)₂, CF₃, OCH₃, OCH₂CH₃, isopropoxy, OCF₃, OCHF₂, NHCH₃, N(CH₃)₂, NHCH₂CH₃ or NHCH₂(CH₃)₂.

In a further embodiment, R^{a} is halogen and preferably selected from F and Cl and in particular, R^{a} is Cl. In a further embodiment, R^{a} is C₁-C₆-alkyl and preferably selected from methyl, ethyl, n-propyl, i-propyl and t-butyl. In a further embodiment, R^{a} is C₁-C₆-haloalkyl. More preferably, R^{a} is C₁-haloalkyl and in particular, R^{a} is trifluormethyl. In a further embodiment, R^{a} is C₁-C₄-alkoxy and preferably selected from methoxy, ethoxy, n-propyloxy and i-propyloxy.

In a further embodiment, two radicals R^{a} that are bound to adjacent ring member atoms of the pyridazine or pyrimidine ring form together with said ring member atoms a fused 5-, 6- or 7-membered saturated, partially unsaturated or aromatic aromatic cycle, which may be a carbocycle or heterocycle, wherein the ring member atoms of the fused heterocycle include besides carbon atoms one, two, three or four heteroatoms selected from the group of N, O and S, and wherein the fused carbocycle or heterocycle is unsubstituted and carries 1, 2, 3 or 4 identical or different groups as defined for R^{a}. In one embodiment, the fused cycle is preferably phenyl. In a another embodiment, the fused cycle is preferably a saturated carbocycle and in particular cyclohexyl. In a further embodiment, the fused cycle is preferably a partially unsaturated carbocycle and in particular cyclohexenyl.

Preference is given to compounds I, wherein two radicals R^{a} that are bound to adjacent ring member atoms of the pyridazine or pyrimidine ring form together with said ring member atoms a fused optionally substituted 6-membered heteroaryl.

Preference is given to compounds I, wherein two radicals R^{a} that are bound to adjacent ring member atoms of the pyridazine or pyrimidine ring form together with said ring member atoms a fused optionally substituted 5-membered heteroaryl.

Specific embodiments relate to compounds I, wherein R^{a1}, R^{a2} and R^{a3} are each independently hydrogen or have one of the definitions specified for R^{a} and wherein the pyridazinyl group carries one of the following combinations of the radicals R^{a1}, R^{a2} and R^{a3} as defined in Table P, which compounds are of formula I.1 or I.2

TABLE P
I.1

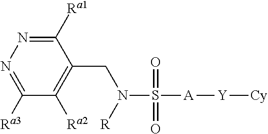

or
I.2

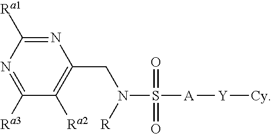

| line | Formula | R^{a1} | R^{a2} | R^{a3} |
|---|---|---|---|---|
| P-1 | I.1 | H | H | H |
| P-2 | I.1 | OCH₃ | H | H |
| P-3 | I.1 | OC₂H₅ | H | H |
| P-4 | I.1 | OCF₃ | H | H |
| P-5 | I.1 | OCHF₂ | H | H |
| P-6 | I.1 | H | H | OCH₃ |
| P-7 | I.1 | H | H | OC₂H₅ |
| P-8 | I.1 | H | H | OCF₃ |
| P-9 | I.1 | H | H | OCHF₂ |
| P-10 | I.1 | OCH₃ | H | OCH₃ |
| P-11 | I.2 | OCH₃ | H | H |
| P-12 | I.2 | OC₂H₅ | H | H |
| P-13 | I.2 | OCF₃ | H | H |
| P-14 | I.2 | OCHF₂ | H | H |

One embodiment relates to compounds I, wherein R is hydrogen, C₁-C₆-alkyl, C₁-C₆-haloalkyl, C₁-C₆-alkylcarbonyl or C₁-C₆-haloalkylcarbonyl, preferably hydrogen or C₁-C₆-alkyl.

Another embodiment relates to compounds I, wherein R is C₁-C₄-alkyl, —CH2—CH═CH₂ or —CH₂——C═CH.

A further embodiment relates to compounds I, wherein R is C₁-C₄-alkyl and preferably selected from methyl, ethyl, n-propyl and i-propyl, and in particular, R is methyl.

A further embodiment relates to compounds I, wherein R is hydrogen and wherein R^{a1}, R^{a2} and R^{a3} are each independently hydrogen or have one of the definitions specified for R^{a}, especially those being preferred, which compounds are of formula I.3 or I.4

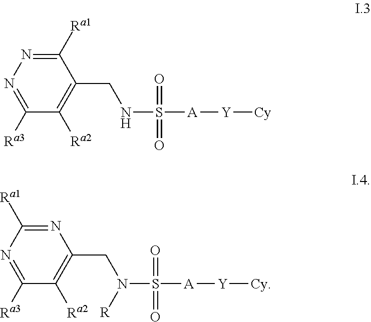

One embodiment of the invention relates to compounds I, wherein A is phenylene or heteroarenediyl, as defined above, which both are unsubstituted or carry 1, 2, 3 or 4 identical or different substituents R^{b}.

Another embodiment relates to compounds I, wherein A is 1,4-phenylene, which is unsubstituted or carries 1, 2, 3 or 4 identical or different substituents R^{b}, in particular A is 1,4-phenylene, which is unsubstituted.

A further embodiment relates to compounds I, wherein A is a heteroarenediyl selected from the group consisting of pyridindiyl, pyrimidindiyl, pyridazindiyl, pyrazindiyl, triazindiyl, furandiyl, thiendiyl, pyrroldiyl, pyrazoldiyl, isoxazoldiyl, isothiazoldiyl, imidazoldiyl, oxazoldiyl, thiazoldiyl, triazoldiyl, thiadiazoldiyl and oxadiazoldiyl, and wherein the aforementioned radicals are unsubstituted or carry 1, 2 or 3 identical or different substituents R^{b}.

Amongst compounds I, wherein A is a 6-membered heteroarenediyl, particular preference given to those, wherein A is pyridindiyl or pyrimidinyl, wherein each of the aforementioned two radicals are unsubstituted or carry 1, 2 or 3 identical or different substituents R^{b}.

Amongst compounds I, wherein A is a 5-membered heteroarenediyl, particular preference given to those, wherein A is thiendiyl, thiazoldiyl, oxazoldiyl, pyrazoldiyl or pyridindiyl, wherein each of the aforementioned five radicals are unsubstituted or carry 1, 2 or 3 identical or different substituents R^{b}.

Particularly preferred embodiments of the invention relate to compounds I, in which A is one of the following radicals A-1 to A-16:

| No. | A |
|---|---|
| A-1 | 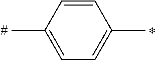 |
| A-2 | 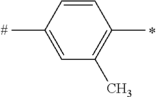 |
| A-3 | 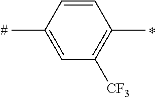 |
| A-4 | 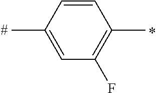 |
| A-5 | 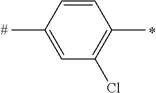 |
| A-6 | 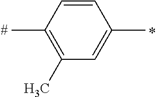 |
| A-7 | 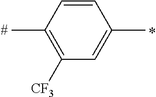 |
| A-8 | 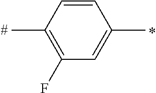 |
| A-9 | 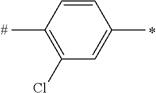 |
| A-10 | 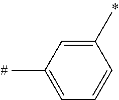 |
| A-11 | 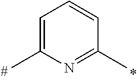 |
| A-12 | 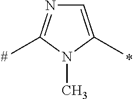 |
| A-13 | 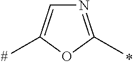 |
| A-14 | 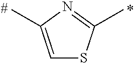 |
| A-15 | 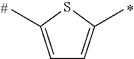 |
| A-16 | 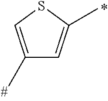 |

wherein # indicates the bond to the sulfur atom of the sufonamide group; and * indicates the bond to Y.

One embodiment of the invention relates to compounds I, wherein the group A carries 1, 2 or 3 radicals R^{b}, more preferably 1 or 2 radicals R^{b}. In another embodiment, the group A is unsubstituted or carries 1 radical R^{b}. In a further embodiment, the group A is unsubstituted. In a further embodiment, the group A carries 1 radical R^{b}. In a further embodiment, the group A carries 2 radicals R^{b}. In a further embodiment, the group A carries 3 radicals R^{b}.

If R^{b} is present, R^{b} is preferably halogen, CN, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, C₁-C₄-alkylcarbonyl, C₁-C₄-alkoxycarbonyl, C₁-C₄-alkylamino, di(C₁-C₄-alkyl)amino, C₁-C₄-alkylaminocarbonyl or di(C₁-C₄-alkyl)aminocarbonyl. More preferably, R^{b} is halogen, CN, C₁-C₄-alkyl, C₁-C₄-haloalkyl, C₁-C₄-alkoxy or C₁-C₄-halo-alkoxy.

In a further embodiment, R^{b} is halogen and preferably selected from fluorine and chlorine, and in particular, chlorine. In a further embodiment, R^{b} is C₁-C₄-alkyl and preferably selected from methyl, ethyl, n-propyl and i-propyl, and in particular, methyl. In a further embodiment, R^{b} is C₁-C₄-haloalkyl.

In a further embodiment, two radicals R^{b} that are bound to adjacent ring member atoms of the group A do not form together with said ring member atoms any fused cycle.

A further embodiment relates to compounds I, wherein two radicals R^{b} that are bound to adjacent ring member atoms of the group A form together with said ring member atoms a fused cycle being a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused cycle is unsubstituted and carries 1, 2, 3 or 4 identical or different groups as defined for R^{b}. In one embodiment, the fused cycle is preferably phenyl. In another embodiment, the fused cycle is preferably a saturated carbocycle and in particular cyclohexyl. In a further embodiment, the fused cycle is preferably a partially unsaturated carbocycle and in particular cyclohexenyl.

One embodiment relates to compounds I, wherein Y is —O—, —S— or —NH—. Another embodiment relates to compounds I, wherein Y is —S— or —O—. A further embodiment relates to compounds I, wherein R is hydrogen and Y is —O— and R^{a1}, R^{a2} and R^{a3} are each independently hydrogen or have one of the definitions specified for R^{a}, which are represented by formula I.A1 and I.A2:

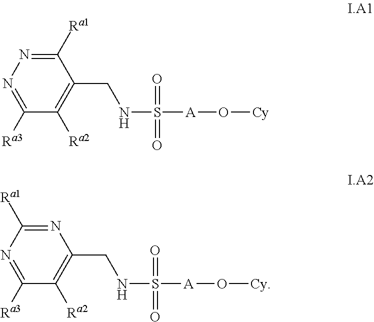

A further embodiment relates to compounds I, wherein Y is —N(R^{Π})—, wherein R^{Π} is hydrogen or C₁-C₄-alkyl. If R^{Π} is present, in one embodiment of the invention, R^{Π} is C₁-C₄-alkyl, and preferably selected from methyl, ethyl, n-propyl and i-propyl, and in particular, R^{Π} is methyl. Particullarly preferred compounds I, wherein R is hydrogen and Y is —N(CH₃)— and R^{a1}, R^{a2} and R^{a3} are each independently hydrogen or have one of the definitions specified for R^{a}, which are represented by formula I.B1 and I.B2.

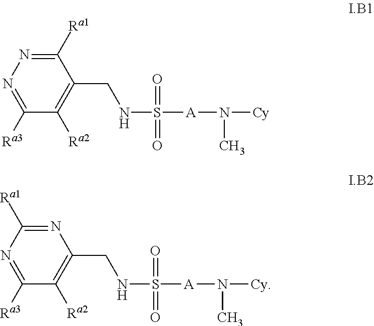

A further embodiment relates to compounds I, wherein R is hydrogen and Y is —NH— and R^{a1}, R^{a2} and R^{a3} are each independently hydrogen or have one of the definitions specified for R^{a}, which are represented by formula I.C1 and I.C2:

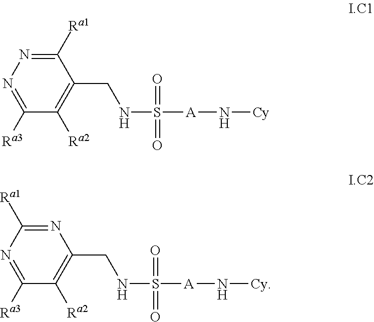

A further embodiment relates to compounds I, wherein R is hydrogen and Y is —S— and R^{a1}, R^{a2} and R^{a3} are each independently H or have one of the definitions specified for R^{a}, which are represented by formula I.D1 and I.D2:

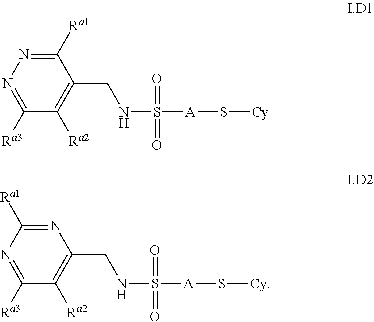

A further embodiment relates to compounds I, wherein R is hydrogen and Y is —S(═O)— and R^{a1}, R^{a2} and R^{a3} are each independently hydrogen or have one of the definitions specified for R^{a}, which are represented by formula I.E1 and I.E2:

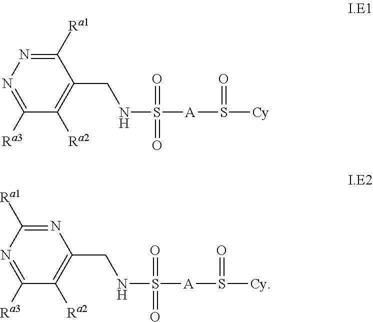

A further embodiment relates to compounds I, wherein R is hydrogen and Y is —S(═O)₂— and R^{a1}, R^{a2} and R^{a3} are each independently hydrogen or have one of the definitions specified for R^{a}, which are represented by formula I.F1 and I.F2:

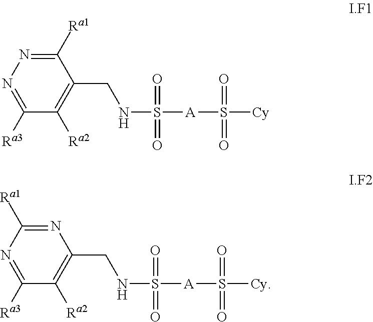

One embodiment relates to compounds I, wherein Cy is C₃-C₁₀-cycloalkyl and prefarably selected from cyclopropyl, cyclopentyl, cyclohexyl, norbornyl and adamantyl, and in particular cyclohexyl, and wherein the aforementioned radicals are unsubstituted or carry 1, 2, 3, 4 or 5 identical or different substituents R^{c}.

Another embodiment relates to compounds I, wherein Cy is cyclopropyl, which cyclopropyl is unsubstituted or carries 1, 2, 3 or 4 identical or different substituents R^{c}.

A further embodiment relates to compounds I, wherein Cy is C₃-C₁₀-cycloalkenyl and preferably selected from cyclopropenyl, cyclopentenyl, cyclohexenyl and norbornenyl, and in particular cyclohexenyl, and wherein the aforementioned radicals are unsubstituted or carry 1, 2, 3, 4 or 5 identical or different substituents R^{c}.

Particularly preferred embodiments of the invention relate to compounds I, in which Cy is one of the following radicals C-1 to C-21:

| No. | Cy |
|---|---|
| C-1 | 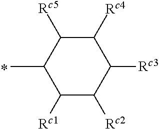 |
| C-2 | 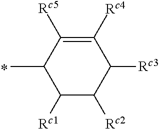 |
| C-3 | 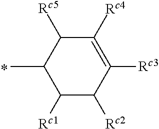 |
| C-4 | 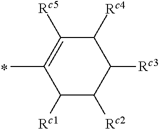 |
| C-5 | 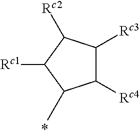 |
| C-6 | 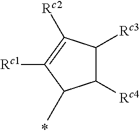 |
| C-7 | 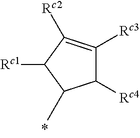 |
| C-8 | 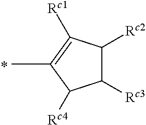 |
| C-9 | 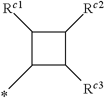 |
| C-10 | 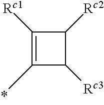 |
| C-11 | 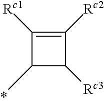 |

in which * indicates the bond to Y; and R^{c1}, R^{c2}, R^{c4} and R^{c5} are each independently hydrogen or have one of the definitions specified for R^{c}, especially those being preferred.

One embodiment of the invention relates to compounds I, wherein Cy carries 1, 2 or 3 radicals R^{c}, preferably Cy carries 1 or 2 radicals R^{c}, in particular Cy carries 1 radical R^{c}. A further embodiment relates to compounds I, wherein Cy carries 2 radicals R^{c}. A further embodiment relates to compounds I, wherein Cy carries 3 radicals R^{c}. A further embodiment relates to compounds I, wherein Cy is unsubstituted. In a further embodiment, two radicals R^{c} that are bound to adjacent ring member atoms of the group Cy do not form together with said ring member atoms any fused cycle.

Preferably, R^{c} is halogen, CN, C₁-C₆-alkyl, C₁-C₆-haloalkyl, C₁-C₆-alkoxy, C₁-C₆-haloalkoxy, C₁-C₆-alkoxy-C₁-C₆-alkyl, C(═O)R′, C(═NOR″)R′″, C₃-C₈-cycloalkyl, C₁-C₆-alkyl-C₃-C₈-cycloalkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 heteroatoms selected from the group of N, O and S, and wherein the aforementioned cyclic radicals are unsubstituted or carry 1, 2, 3 or 4 identical or different substituents R^{d}.

In one embodiment, R^{c} is halogen and preferably selected from F and Cl and in particular, R^{c} is Cl. In another embodiment, R^{c} is CN. In a further embodiment, R^{c} is C₁-C₆-alkyl and preferably selected from methyl, ethyl, n-propyl and i-propyl, and in particular, R^{c} is methyl. In a further embodiment, R^{c} is C₁-C₆-haloalkyl. More preferably, R^{c} is C₁-haloalkyl and selected from fluoromethyl, difluoromethyl, trifluoromethyl, chloromethyl, dichioromethyl and trichloromethyl, and in particular, R^{c} is trifluoromethyl. In a further embodiment, R^{c} is C₁-C₆-alkoxy and preferbly selected from methoxy and ethoxy. In a further embodiment, R^{c} is C₁-C₆-haloalkoxy and preferably halomethoxy such as difluormethoxy, trifluormethoxy, dichlormethoxy and trichlormethoxy; halo-ethoxy such as 2,2-difluorethoxy, 2,2,2-trifluorethoxy, 2,2-dichlorethoxy and 2,2,2-trichloroethoxy; halo-n-propoxy, halo-i-propoxy, halo-n-butoxy, halo-1-methyl-propoxy, halo-2-methyl-propoxy or halo-1,1-dimethylethoxy. In a further embodiment, R^{c} is C₁-C₆-alkoxy-C₁-C₆-alkyl and preferably selected from methoxymethyl, ethoxymethyl, methoxyethyl and ethoxyethyl.

A skilled person will readily understand that the preferences given in connection with compounds I apply for formulae I.1, I.2, I.3 and I.4 and formulae I.A1 to I.F2 as defined above.

With respect to their use, particular preference is given to the compounds of formulae I.A1 to I.F2 compiled in the tables 1 to 2688 below, wherein the definitions for the substituents R^{a} of the pyridine group are selected from P-1 to P-14 in Table P and wherein the definitions for group A are selected from A-1 to A-16 as described above and wherein the definitions for group Cy are selected from C-1 to C-11. Here, the groups mentioned in the Tables for a substituent are furthermore, independently of the combination wherein they are mentioned, a particularly preferred embodiment of the substituent in question.
Table 1: Compounds of formula I.A1, wherein R^{a1}, R^{a2} and R^{a3} are defined as in line P-1 of table P, A is A-1 as defined before and the meaning of Cy for each compound corresponds to one line of table A.
Table 2: Compounds of formula I.A1, wherein R^{a1}, R^{a2} and R^{a3} are defined as in line P-2 of table P, A is A-1 as defined before and the meaning of Cy for each compound corresponds to one line of table A.
Table 3: Compounds of formula I.A1, wherein R^{a1}, R^{a2} and R^{a3} are defined as in line P-3 of table P, A is A-1 as defined before and the meaning of Cy for each compound corresponds to one line of table A.
Table 4: Compounds of formula I.A1, wherein R^{a1}, R^{a2} and R^{a3} are defined as in line P-4 of table P, A is A-1 as defined before and the meaning of Cy for each compound corresponds to one line of table A.
Table 5: Compounds of formula I.A1, wherein R^{a1}, R^{a2} and R^{a3} are defined as in line P-5 of table P, A is A-1 as defined before and the meaning of Cy for each compound corresponds to one line of table A.
Table 6: Compounds of formula I.A1, wherein R^{a1}, R^{a2} and R^{a3} are defined as in line P-6 of table P, A is A-1 as defined before and the meaning of Cy for each compound corresponds to one line of table A.
Table 7: Compounds of formula I.A1, wherein R^{a1}, R^{a2} and R^{a3} are defined as in line P-7 of table P, A is A-1 as defined before and the meaning of Cy for each compound corresponds to one line of table A.
Table 8: Compounds of formula I.A1, wherein R^{a1}, R^{a2} and R^{a3} are defined as in line P-8 of table P, A is A-1 as defined before and the meaning of Cy for each compound corresponds to one line of table A.
Table 9: Compounds of formula I.A1, wherein R^{a1}, R^{a2} and R^{a3} are defined as in line P-9 of table P, A is A-1 as defined before and the meaning of Cy for each compound corresponds to one line of table A.
Table 10: Compounds of formula I.A1, wherein R^{a1}, R^{a2} and R^{a3} are defined as in line P-10 of table P, A is A-1 as defined before and the meaning of Cy for each compound corresponds to one line of table A. Table 11: Compounds of formula I.A1, wherein R^{a1}, R^{a2} and R^{a1} are defined as in line P-11 of table P, A is A-1 as defined before and the meaning of Cy for each compound corresponds to one line of table A.

Table 12: Compounds of formula I.A1, wherein R^{a1}, R^{a2} and R^{a3} are defined as in line P-12 of table P, A is A-1 as defined before and the meaning of Cy for each compound corresponds to one line of table A.
Table 13: Compounds of formula 1.A1, wherein R^{a1}, R^{a2} and R^{a3} are defined as in line P-13 of table P, A is A-1 as defined before and the meaning of Cy for each compound corresponds to one line of table A.
Table 14: Compounds of formula I.A1, wherein R^{a1}, R^{a2} and R^{a3} are defined as in line P-14 of table P, A is A-1 as defined before and the meaning of Cy for each compound corresponds to one line of table A.
Tables 15 to 28: Compounds of formula I.A1, wherein R^{a1}, R^{a2} and R^{a3} are defined as in Tables 1 to 80, A is A-2 instead of A-1 and the meaning of Cy for each compound corresponds to one line of table A.
Tables 29 to 42: Compounds of formula I.A1, wherein R^{a1}, R^{a2} and R^{a3} are defined as in Tables 1 to 80, A is A-3 instead of A-1 and the meaning of Cy for each compound corresponds to one line of table A.
Tables 43 to 56: Compounds of formula I.A1, wherein R^{a1}, R^{a2} and R^{a3} are defined as in Tables 1 to 80, A is A-4 instead of A-1 and the meaning of Cy for each compound corresponds to one line of table A.
Tables 57 to 70: Compounds of formula I.A1, wherein R^{a1}, R^{a2} and R^{a3} are defined as in Tables 1 to 80, A is A-5 instead of A-1 and the meaning of Cy for each compound corresponds to one line of table A.
Tables 71 to 84: Compounds of formula I.A1, wherein R^{a1}, R^{a2} and R^{a3} are defined as in Tables 1 to 80, A is A-6 instead of A-1 and the meaning of Cy for each compound corresponds to one line of table A.
Tables 85 to 98: Compounds of formula I.A1, wherein R^{a1}, R^{a2} and R^{a3} are defined as in Tables 1 to 80, A is A-7 instead of A-1 and the meaning of Cy for each compound corresponds to one line of table A.
Tables 99 to 112: Compounds of formula I.A1, wherein R^{a1}, R^{a2} and R^{a3} are defined as in Tables 1 to 80, A is A-8 instead of A-1 and the meaning of Cy for each compound corresponds to one line of table A.
Tables 113 to 126: Compounds of formula I.A1, wherein R^{a1}, R^{a2} and R^{a3} are defined as in Tables 1 to 80, A is A-9 instead of A-1 and the meaning of Cy for each compound corresponds to one line of table A.
Tables 127 to 140: Compounds of formula I.A1, wherein R^{a1}, R^{a2} and R^{a3} are defined as in Tables 1 to 80, A is A-10 instead of A-1 and the meaning of Cy for each compound corresponds to one line of table A.
Tables 141 to 154: Compounds of formula I.A1, wherein R^{a1}, R^{a2} and R^{a3} are defined as in Tables 1 to 80, A is A-11 instead of A-1 and the meaning of Cy for each compound corresponds to one line of table A.
Tables 155 to 168: Compounds of formula I.A1, wherein R^{a1}, R^{a2} and R^{a3} are defined as in Tables 1 to 80, A is A-12 instead of A-1 and the meaning of Cy for each compound corresponds to one line of table A.
Tables 169 to 182: Compounds of formula I.A1, wherein R^{a1}, R^{a2} and R^{a3} are defined as in Tables 1 to 80, A is A-13 instead of A-1 and the meaning of Cy for each compound corresponds to one line of table A. as in Tables 1 to 80, A is A-14 instead of A-1 and the meaning of Cy for each compound corresponds to one line of table A.
Tables 197 to 210: Compounds of formula I.A1, wherein R^{a1}, R^{a2} and R^{a3} are defined as in Tables 1 to 80, A is A-15 instead of A-1 and the meaning of Cy for each compound corresponds to one line of table A.
Tables 211 to 224: Compounds of formula I.A1, wherein R^{a1}, R^{a2} and R^{a3} are defined as in Tables 1 to 80, A is A-16 instead of A-1 and the meaning of Cy for each compound corresponds to one line of table A.
Tables 225 to 448: Compounds of formula I.A2, wherein A, R^{a1}, R^{a2} and R^{a1} are defined as in Tables 1 to 224 and the meaning of Cy for each compound corresponds to one line of table A.
Tables 449 to 672: Compounds of formula I.B1, wherein A, R^{a1}, R^{a2} and R^{a3} are defined as in Tables 1 to 224 and the meaning of Cy for each compound corresponds to one line of table A.
Tables 673 to 896: Compounds of formula I.B2, wherein A, R^{a1}, R^{a2} and R^{a3} are defined as in Tables 1 to 224 and the meaning of Cy for each compound corresponds to one line of table A.
Tables 897 to 1120: Compounds of formula I.C1, wherein A, R^{a1}, R^{a2} and R^{a3} are defined as in Tables 1 to 224 and the meaning of Cy for each compound corresponds to one line of table A.
Tables 1121 to 1344: Compounds of formula I.C2, wherein A, R^{a1}, R^{a2} and R^{a3} are defined as in Tables 1 to 224 and the meaning of Cy for each compound corresponds to one line of table A.
Tables 1345 to 1568: Compounds of formula I.D1, wherein A, R^{a1}, R^{a2} and R^{a3} are defined as in Tables 1 to 224 and the meaning of Cy for each compound corresponds to one line of table A.
Tables 1569 to 1792: Compounds of formula I.D2, wherein A, R^{a1}, R^{a2} and R^{a1} are defined as in Tables 1 to 224 and the meaning of Cy for each compound corresponds to one line of table A.
Tables 1793 to 2016: Compounds of formula I.E1, wherein A, R^{a1}, R^{a2} and R^{a3} are defined as in Tables 1 to 224 and the meaning of Cy for each compound corresponds to one line of table A.
Tables 2017 to 2240: Compounds of formula I.E2, wherein A, R^{a1}, R^{a2} and R^{a3} are defined as in Tables 1 to 224 and the meaning of Cy for each compound corresponds to one line of table A.
Tables 2241 to 2464: Compounds of formula I.F1, wherein A, R^{a1}, R^{a2} and R^{a3} are defined as in Tables 1 to 224 and the meaning of Cy for each compound corresponds to one line of table A.
Tables 2465 to 2688: Compounds of formula I.F2, wherein A, R^{a1}, R^{a2} and R^{a3} are defined as in Tables 1 to 224 and the meaning of Cy for each compound corresponds to one line of table A.

TABLE A

| line | Cy | R^{c1} | R^{c2} | R^{c3} | R^{c4} | R^{c5} |
|---|---|---|---|---|---|---|
| 1 | C-1 | H | H | H | H | H |
| 2 | C-1 | CH₃ | H | H | H | H |
| 3 | C-1 | H | CH₃ | H | H | H |
| 4 | C-1 | H | H | CH₃ | H | H |
| 5 | C-1 | CH₃ | CH₃ | H | H | H |
| 6 | C-1 | CH₃ | H | CH₃ | H | H |
| 7 | C-1 | CH₃ | H | H | CH₃ | H |
| 8 | C-1 | CH₃ | H | H | H | CH₃ |
| 9 | C-1 | H | CH₃ | CH₃ | H | H |
| 10 | C-1 | H | CH₃ | H | CH₃ | H |
| 11 | C-2 | H | H | H | H | H |
| 12 | C-2 | CH₃ | H | H | H | H |
| 13 | C-2 | H | CH₃ | H | H | H |
| 14 | C-2 | H | H | CH₃ | H | H |
| 15 | C-3 | H | H | H | H | H |
| 16 | C-3 | CH₃ | H | H | H | H |
| 17 | C-3 | H | CH₃ | H | H | H |
| 18 | C-3 | H | H | CH₃ | H | H |
| 19 | C-4 | H | H | H | H | H |
| 20 | C-4 | CH₃ | H | H | H | H |
| 21 | C-5 | H | H | H | H | |
| 22 | C-5 | CH₃ | H | H | H | |
| 23 | C-5 | H | H | CH₃ | H | |
| 24 | C-6 | H | H | H | H | |
| 25 | C-6 | CH₃ | H | H | H | |
| 26 | C-6 | H | CH₃ | H | H | |
| 27 | C-6 | H | H | CH₃ | H | |
| 28 | C-6 | H | H | H | CH₃ | |
| 29 | C-7 | H | H | H | H | |
| 30 | C-7 | CH₃ | H | H | H | |
| 31 | C-7 | H | CH₃ | H | H | |
| 32 | C-7 | H | H | CH₃ | H | |
| 33 | C-7 | H | H | H | CH₃ | |
| 34 | C-8 | H | H | H | H | |
| 35 | C-8 | CH₃ | H | H | H | |
| 36 | C-8 | H | CH₃ | H | H | |
| 37 | C-9 | H | H | H | | |
| 38 | C-9 | CH₃ | H | H | | |
| 39 | C-9 | H | CH₃ | H | | |
| 40 | C-9 | H | H | CH₃ | | |
| 41 | C-10 | H | H | H | | |
| 42 | C-10 | CH₃ | H | H | | |
| 43 | C-10 | H | CH₃ | H | | |
| 44 | C-10 | H | H | CH₃ | | |
| 45 | C-11 | H | H | H | | |
| 46 | C-11 | CH₃ | H | H | | |
| 47 | C-11 | H | CH₃ | H | | |

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

The compounds I and compositions thereof, respectively, are particularly suitable for controlling the following plant diseases: Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e.g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines; Erysiphe spp. (powdery mildew) on sugar beets (E. betae), vegetables (e. g. E. pisi), such as cucurbits (e. g. E. cichoracearum), cabbages, rape (e. g. E. cruciferarum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani on soybeans and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Guignardia bidwellii (black rot) on vines; Microdochium (syn. Fusarium) nivale (pink snow mold) on cereals (e. g. wheat or barley); Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight); Plasmopara spp., e. g. P. viticola (grapevine downy mildew) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, and asparagus (e. g. P. asparagi); Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhynchosporium secalis (scald) on barley, rye and triticale; Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Stagonospora.spp. on cereals, e. g. S. nodorum (Stagono-spora blotch, teleomorph: Leptosphaeria [syn. Phaeosphaeria] nodorum) on wheat; Venturia spp. (scab) on apples (e. g. V. inaequalis) and pears.

The compounds I and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, coiling lubricants, fiber or fabrics, against the infestation and destruction by harmful microorganisms, such as fungi and bacteria.

The compounds I and compositions thereof, resepectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

The invention also relates to agrochemical compositions comprising a solvent or solid carrier and at least one compound I and to the use for controlling harmful fungi.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF).

Usually the composition types (e. g. SC, OD, FS, EC, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, S. 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e. g. for seed treatment formulations).

Powders, materials for spreading and dusts can be prepared by mixing or concomitantly grinding the compounds I and, if appropriate, further active substances, with at least one solid carrier.

Granules, e. g. coated granules, impregnated granules and homogeneous granules, can be prepared by binding the active substances to solid carriers.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations.

In a preferred embodiment, a suspension-type (FS) composition is used for seed treatment. Typcially, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives. When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are, e. g., 0.001 g to 2 kg, preferably 0.005 g to 1 kg, of active substance per cubic meter of treated material.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

The compositions according to the invention can, in the use form as fungicides, also be present together with other active substances, e. g. with herbicides, insecticides, growth regulators, fungicides or else with fertilizers, as pre-mix or, if appropriate, not until immeadiately prior to use (tank mix).

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds according to the invention can be used, is intended to illustrate the possible combinations but does not limit them:
A) strobilurins
   azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)-phenyl)-2-methoxy-imino-N-methyl-acetamide, 3-methoxy-2-(2-(N-(4-methoxy-phenyl)-cyclopropane-carboximidoylsulfanylmethyl)-phenyl)-acrylic acid methyl ester, methyl (2-chloro-5-[1-(3-methylbenzyloxyimino)ethyl]benzyl)carbamate and 2-(2-(3-(2,6-di-chlorophenyl)-1-methyl-allylideneaminooxymethyl)-phenyl)-2-methoxyimino-N-methyl-acetamide;
B) carboxamides
   carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methyl-thiazole-5-carboxanilide, 2-chloro-N-(1,1,3-trimethyl-indan-4-yl)-nicotinamide, N-(3′,4′,5′-trifluorobiphenyl-2-yl)-3-difluoromethyl-1-methyl-1H-pyrazole-4-carboxamide, N-(4′-trifluoro-methylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide and N-(2-(1,3,3-trimethyl-butyl)-phenyl)-1,3-dimethyl-5-fluoro-1H-pyrazole-4-carbox-amide;
   carboxylic morpholides: dimethomorph, flumorph, pyrimorph;
   benzoic acid amides: flumetover, fluopicolide, fluopyram, zoxamide, N-(3-Ethyl-3,5,5-trimethyl-cyclohexyl)-3-formylamino-2-hydroxy-benzamide;
   other carboxamides: carpropamid, dicyclomet, mandiproamid, oxytetracyclin, silthiofarm and N-(6-methoxy-pyridin-3-yl) cyclopropanecarboxylic acid amide;
C) azoles
   triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([l,2,4]triazol-1-yl)-cycloheptanol; imidazoles: cyazofamid, imazalil, pefurazoate, prochloraz, triflumizol; benzimidazoles: benomyl, carbendazim, fuberidazole, thiabendazole; others: ethaboxam, etridiazole, hymexazole and 2-(4-chloro-phenyl)-N-[4-(3,4-di-methoxy-phenyl)isoxazol-5-yl]-2-prop-2-ynyloxy-acetamide;
D) heterocyclic compounds
   pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbo-nitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;
   pyrimidines: bupirimate, cyprodinil, diflumetorim, fenarimol, ferimzone, mepanipyrim, nitrapyrin, nuarimol, pyrimethanil;
   piperazines: triforine;
   pyrroles: fenpiclonil, fludioxonil;
   morpholines: aldimorph, dodemorph, dodernorph-acetate, fenpropimorph, tridemorph;
   piperidines: fenpropidin;
   dicarboximides: fluoroimid, iprodione, procymidone, vinclozolin;
   non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester;
   others: acibenzolar-S-methyl, amisulbrom, anilazin, blasticidin-S, captafol, captan, chinomethionat, dazomet, debacarb, diclomezine, difenzoquat, difenzoquat-methyl-sulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, 5-chloro-7-(4-methyl-piperidin-1-yl)-6-(2,4,6-trifluorophenyl)-[1,2,4]triazolo[1,5-a]pyrimidine and 5-ethyl-6-octyl-[1,2,4]triazolo[1,5-a]pyrimidine-7-ylamine;
E) carbamates
   thio- and dithiocarbamates: ferbam, mancozeb, maneb, metam, methasulphocarb, metiram, propineb, thiram, zineb, ziram;
   carbamates: benthiavalicarb, diethofencarb, iprovalicarb, propamocarb, propamo-carb hydrochlorid, valiphenal and N-(1-(1-(4-cyano-phenyl)ethanesulfonyl)-but-2-yl) carbamic acid-(4-fluorophenyl) ester;
F) other active substances
   guanidines: guanidine, dodine, dodine free base, guazatine, guazatine-acetate, iminoctadine, iminoctadine-triacetate, iminoctadine-tris(albesilate);
   antibiotics: kasugamycin, kasugamycin hydrochloride-hydrate, streptomycin, polyoxine, validamycin A;
   nitrophenyl derivates: binapacryl, dinobuton, dinocap, nitrthal-isopropyl, tecnazen, organometal compounds: fentin salts, such as fentin-acetate, fentin chloride or fentin hydroxide;
   sulfur-containing heterocyclyl compounds: dithianon, isoprothiolane;
   organophosphorus compounds: edifenphos, fosetyl, fosetyl-aluminum, iprobenfos, phosphorous acid and its salts, pyrazophos, tolclofos-methyl;
   organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide;
   inorganic active substances: Bordeaux mixture, copper acetate, copper hydroxide, copper oxychloride, basic copper sulfate, sulfur;
   others: biphenyl, bronopol, cyflufenamid, cymoxanil, diphenylamin, metrafenone, mildiomycin, oxin-copper, prohexadione-calcium, spiroxamine, tolylfluanid, N-(cyclo-propylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethyl-silanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1[2-(5-meth-yl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, acetic acid 6-tert.-butyl-8-fluoro-2,3-dimethyl-quinolin-4-ylester, methoxy-acetic acid 6-tert-butyl-8-fluoro-2,3-dimethyl-quinolin-4-yl ester and N-methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thi-azolecarboxamide.

The present invention furthermore relates to agrochemical compositions comprising a mixture of at least one compound I (component 1) and at least one further active substance useful for plant protection, e. g. selected from the groups A) to I) (component 2), in particular one further fungicide, e. g. one or more fungicide from the groups A) to F), as described above, and if desired one suitable solvent or solid carrier. Those mixtures are of particular interest, since many of them at the same application rate show higher efficiencies against harmful fungi. By applying compounds I together with at least one active substance from groups A) to I) a synergistic effect can be obtained, i.e. more then simple addition of the individual effects is obtained (synergistic mixtures).

In binary mixtures, i.e. compositions according to the invention comprising one compound I (component 1) and one further active substance (component 2), e. g. one active substance from groups A) to I), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary mixtures, i.e. compositions according to the invention comprising one compound I (component 1) and a first further active substance (component 2) and a second further active substance (component 3), e. g. two active substances from groups A) to I), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

Preference is also given to mixtures comprising a compound I (component 1) and at least one active substance selected from the strobilurines of group A) (component 2) and particularly selected from azoxystrobin, dimoxystrobin, fluoxastrobin, kresoxim-methyl, orysastrobin, picoxystrobin, pyraclostrobin and trifloxystrobin.

Preference is also given to mixtures comprising a compound I (component 1) and at least one active substance selected from the carboxamides of group B) (component 2) and particularly selected from bixafen, boscalid, sedaxane, fenhexamid, metalaxyl, isopyrazam, mefenoxam, ofurace, dimethomorph, flumorph, fluopicolid (picobenzamid), zoxamide, carpropamid, mandipropamid and N-(3′,4′,5′-trifluorobiphenyl-2-yl)-3-di-fluoromethyl-1-methyl-1H-pyrazole-4-carboxamide. Preference is given to mixtures comprising a compound of formula I (component 1) and at least one active substance selected from the azoles of group C) (component 2) and particularly selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to mixtures comprising a compound I (component 1) and at least one active substance selected from the heterocyclic compounds of group D) (component 2) and particularly selected from fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, proquinazid, acibenzolar-S-methyl, captafol, folpet, fenoxanil, quinoxyfen and 5-ethyl-6-octyl-[1,2,4]triazolo[1,5-a]pyrimidine-7-ylamine.

Preference is also given to mixtures comprising a compound I (component 1) and at least one active substance selected from the carbamates of group E) (component 2) and particularly selected from mancozeb, metiram, propineb, thiram, iprovalicarb, benthiavalicarb and propamocarb.

Preference is also given to mixtures comprising a compound I (component 1) and at least one active substance selected from the fungicides given in group F) (component 2) and particularly selected from dithianon, fentin salts, such as fentin acetate, fosetyl, fosetyl-aluminium, H₃PO₃ and salts thereof, chlorthalonil, dichlofluanid, thiophanat-methyl, copper acetate, copper hydroxide, copper oxychloride, copper sulfate, sulfur, cymoxanil, metrafenone, spiroxamine and N-methyl-2-{1-[(5-methyl-3-trifluoromethyl-1H-pyrazol-1-yl)-acetyl]-piperidin-4-yl}-N-[(1 R)-1,2,3,4-tetrahydronaphthalen-1-yl]-4-thiazolecarboxamide.

The active substances referred to as component 2, their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The mixtures of active substances can be prepared as compositions comprising besides the active ingredients at least one inert ingredient by usual means, e. g. by the means given for the compositions of compounds I. Concerning usual ingredients of such compositions reference is made to the explanations given for the compositions containing compounds I. The mixtures of active substances according to the present invention are suitable as fungicides, as are the compounds of formula I.

### I. SYNTHESIS EXAMPLES

With due modification of the starting compounds, the procedures shown in the synthesis examples below were used to obtain further compounds I.

### Example 1

### Preparation of 4-Cyclopentyloxy-N-(2-Methoxy-Pyrimidin-4-Ylmethyl)-Benzenesulfonamide

A solution of 2-methoxy-pyrimidin-4-yl-methylamine (200 mg) and DIPEA (928mg) in DCM (10 ml) was cooled to about 0° C. and a solution of 5-cyclopentyloxybenzene-sulfonylchloride (356 mg) in DCM (10 ml) was slowly added. After stirring for 14 h, the crude product was adsorbed to silica and purified by means of column chromatography and the title compound obtained as a white solid (30%). Melting point: 111° C.

### Example 2

### Preparation of 4-Cyclopentyloxy-N-(3,6-Dimethoxy-Pyridazin-4-Ylmethyl)-Benzenesulfonamide

A solution of 3,6-dimethoxy-pyridazin-4-yl-methylamine (250 mg) and DIPEA (954 mg) in DCM (10 ml) was cooled to about 0° C. and a solution of 5-cyclopentyloxy-benzenesulfonylchloride (366 mg) in DCM (10 ml)was slowly added. After stirring for 14 h, the crude product was adsorbed to silica and purified by means of column chromato-graphy and the title compound obtained as a white solid (54%). Melting point: 148° C.

1. A compound of formula I 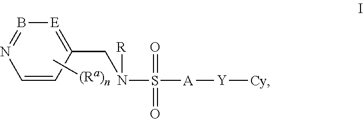
wherein:
B, E are N or CH or C—R^{a} with the provisos that one of B and E must be N and one of B and E must be CH or C—R^{a};
R^{a} is halogen, CN, NH₂, NO₂, OH, SH, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-haloalkylamino, di(C₁-C₆-alkyl)amino, di(C₁-C₆-haloalkyl)amino, C₁-C₆-alkylaminocarbonyl, di(C₁-C₆-alkyl)aminocarbonyl, C₁-C₆-alkoxy-C₁-C₆-alkyl, C₂-C₆-alkenyl, C₂-C₆-haloalkenyl, C₂-C₆-alkynyl, C₂-C₆-haloalkynyl, C₃-C₈-cycloalkyl or C₁-C₆-alkyl-C₃-C₈-cycloalkyl; and/or
two radicals R^{a} that are bound to adjacent ring member atoms of the pyridazine or pyrimidine ring may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group consisting of N, O and S, and wherein the fused carbocycle or heterocycle is unsubstituted or carries 1, 2, 3 or 4 identical or different groups as defined for R^{a};
n indicates the number of the substituents R^{a} on the pyridazine or pyrimidine ring and n is 0, 1, 2 or 3, wherein R^{a} are identical or different if n is 2 or 3;
R is hydrogen, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylcarbonyl, C₁-C₆-haloalkyl-carbonyl, C₁-C₆-alkoxy-C₁-C₆-alkyl, C₁-C₆-haloalkoxy-C₁-C₆-alkyl, C₂-C₆-alkenyl, C₂-C₆-haloalkenyl, C₂-C₆-alkynyl, C₃-C₈-cycloalkyl, cycloalkyl or benzyl, wherein the phenyl moiety of benzyl is unsubstituted or carries 1, 2, 3, 4, or 5 substituents selected from the group consisting of CN, halogen, C₁-C₆-alkyl, C₁-C₆-haloalkyl, C₁-C₆-alkoxy, C₁-C₆-haloalkoxy, C₁-C₆-alkylcarbonyl, C₁-C₆-haloalkylcarbonyl, C₁-C₆-alkoxycarbonyl and di(C₁-C₆-alkyl)aminocarbonyl;
A is C₁-C₈-alkanediyl, C₁-C₆-haloalkanediyl, C₂-C₆-alkenediyl, C₂-C₆-halo-alkenediyl, C₂-C₆-alkynediyl, C₂-C₆-haloalkynediyl, phenylene or a 5- or 6-membered heteroarenediyl, wherein the ring member atoms of the heteroarenediyl include besides carbon atoms 1, 2, 3 or 4 heteroatoms selected from the group consisting of N, O and S, and wherein the aforementioned divalent radicals are unsubstituted or carry 1, 2, 3 or 4 identical or different groups R^{b}:
R^{b} is halogen, CN, NO₂, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, (C₁-C₆-alkyl)carbonyl, (C₁-C₆-alkoxy)carbonyl, C₁-C₆-alkylamino, di(C₁-C₆-alkyl)amino, (C₁-C₆-alkyl)aminocarbonyl or di(C₁-C₆-alkyl)aminocarbonyl;
if A is a cyclic divalent radical, two radicals R^{b} that are bound to adjacent ring member atoms of the group A may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group consisting of N, O and S, and wherein the fused carbocycle or heterocycle is unsubstituted or carries 1, 2, 3 or 4 identical or different groups as defined for R^{b};
Y is a divalent group selected from the group consisting of —O—, —OCH₂—, —CH₂O—, —S—, —S(═O)—, —S(═O)₂—, —N(Rⁿ)— and —C(NOR^{Π})—;
R^{Π} is hydrogen or C₁-C₆-alkyl;
Cy is C₃-C₁₀-cycloalkyl or C₃-C₁₀-cycloalkenyl, which are unsubstituted or carry 1, 2, 3, 4 or 5 identical or different groups R^{c}:
R^{c} is halogen, CN, NO₂, NH₂, C₁-C₆-alkyl, C₁-C₆-alkoxy, C₁-C₆-haloalkoxy, C₁-C₆-alkylamino, di(C₁-C₆-alkyl)amino, C₁-C₆-alkyl-thio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₂-C₆-alkenyl, C₂-C₆-alkynyl, C(═O)R′, C(═NOR″)R′″, C₃-C₈-cycloalkyl, C₁-C₆-alkyl-C₃-C₈-cycloalkyl, phenyl, phenoxy, phenoxy-C₁-C₆-alkyl or a 5- or 6-membered heteroaryl, wherein the ring member atoms of the heteroaryl include besides carbon atoms 1, 2, 3 or 4 heteroatoms selected from the group consisting of N, O and S, and wherein the aforementioned cyclic radicals are unsubstituted or carry 1, 2, 3 or 4 identical or different substituents R^{d}:
R′ is hydrogen, NH₂, C₁-C₆-alkyl, C₂-C₆-alkenyl, C₂-C₆-alkynyl, C₁-C₆-alkoxy, C₁-C₆-alkoxy-C₁-C₆-alkoxy, C₁-C₆-haloalkoxy, C₁-C₆-alkylamino or di(C₁-C₆-alkyl)amino;
R″ is hydrogen, C₁-C₆-alkyl, C₁-C₆-haloalkyl, C₂-C₆-alkenyl, C₂-C₆-alkynyl or C₁-C₆-alkoxy-C₁-C₆-alkyl,
R′″ is hydrogen or C₁-C₆-alkyl;
R^{d} is halogen, CN, C₁-C₆-alkyl, C₁-C₆-alkoxy or C₁-C₆-haloalkoxy;
and/or two radicals R^{c} that are bound to adjacent ring member atoms of the group Het may form together with said ring member atoms a fused 5-, 6- or 7-membered saturated, partially unsaturated or aromatic aromatic cycle, which may be a carbocycle or heterocycle, wherein the ring member atoms of the fused heterocycle include besides carbon atoms 1, 2, 3 or 4 heteroatoms selected from the group consisting of N, O and S, and wherein the fused carbocycle or heterocycle is unsubstituted or carries 1, 2, 3 or 4 identical or different groups R^{e}:
R^{e} is halogen, CN, C₁-C₆-alkyl, C₁-C₆-haloalkyl, C₁-C₆-alkoxy or C₁-C₆-haloalkoxy;
and its N-oxides and agriculturally acceptable salts thereof.

2. The compound of formula I according to claim 1, wherein Cy is cyclopentyl or cyclohexyl.

3. The compound of formula I according to claim 1, wherein A is phenylene.

4. The compound of formula I according to claim 1, wherein Y is —O—.

5. A process for preparing a compound of formula I as defined in claim 1, which comprises reacting a compound of formula II 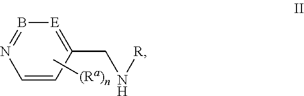
under basic conditions with a sulfonic acid derivative of formula III 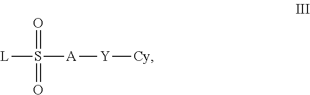
wherein L is a nucleophilic leaving group.

6. A process for preparing a compound of formula I as defined in claim 1, which comprises reacting a compound of formula IV 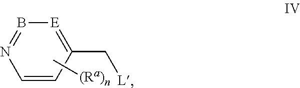
wherein L′ is a leaving group, under basic conditions with compound III.a

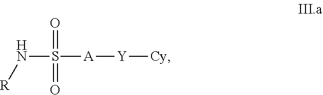

7. An agricultural composition comprising a solvent or solid carrier and at least a compound of formula I or an N-oxide or an agriculturally acceptable salt thereof, according to claim 1 4.

8. The composition according to claim 7 comprising at least one further active substance.

9. A method for combating phytopathogenic fungi, which process comprises treating the fungi or the materials, plants, the soil or seeds to be protected against fungal attack, with an effective amount of at least one compound of claim 1.

10. A seed treated with a compound of claim 1, in an amount of from 0.1 g to 10 kg per 100 kg of seed.

11. The method according to claim 9, wherein Cy is cyclopentyl or cyclohexyl.

12. The method according to claim 9, wherein A is phenylene.

13. The method according to claim 9, wherein Y is —O—.

14. The seed according to claim 10, wherein Cy is cyclopentyl or cyclohexyl.

15. The seed according to claim 10, wherein A is phenylene.

16. The seed according to claim 10, wherein Y is —O—.
